(19) United States
(12) Patent Application Publication
(10) Pub. No.: US 2002/0028445 A1
(43) Pub. Date: Mar. 7, 2002

(54) METHOD AND DEVICE FOR DETECTING THE TOXIC AND MUTAGENIC EFFECT OF CHEMICALS AND MIXTURES OF SUBSTANCES

(76) Inventor: HORST BERCHER, GREIFSWALD (DE)

Correspondence Address: HORST M KASPER, 13 FOREST DRIVE, WARREN, NJ, 07059

(21) Appl. No.: 09/284,682

(22) PCT Filed: Aug. 14, 1998
(86) PCT No.: PCT/DE98/02466

(30) Foreign Application Priority Data
Aug. 15, 1997 (DE) 197 36 261.3

(51) Int. Cl. C12Q 1/68; C12Q 1/02; C12N 1/38

(52) U.S. Cl. 435/6; 435/29; 435/244

(57) ABSTRACT

The invention relates to a method and device for detecting the toxic and mutagenic effect of chemicals and mixtures of substances using immobilized luminescent bacteria or microorganisms that have been rendered bioluminescent by the transfer of adequate vector plasmids, which are provided with an optoelectronic component for photometry in such a way that a biosensor is formed. Since the detection of biological effects can take place during a long period of time, the invention makes it possible to carry out surveillance tasks in the environment and to conduct process controls in agriculture and industry.

### DESCRIPTION

1. Field of the Invention

The invention present as well a procedure as an device to determine the toxic and mutagenic effects of chemicals and mixtures of substances either with the help of immobilized luminescent bacteria or by microorganisms genetically-engineered to phosphoresce due to transfers of appropriate plasmid vectors. For the determination of the biological effects is possible over a long time period, the present invention allows to take over tasks in monitoring environmental parameters and control processes in agriculture and industry.

2. Description of the Background Art

The use of microorganisms and isolated cells for the determination of toxic and mutagenic effects is well-known. To prove the toxic effect of noxious material, bacteria (DE-OS 3902982; LÜMMEN, P.: forum mikrobiologie 10, 428-434 (1988)), isolated cells (ROSSI, A. et al.: Pharmacol. & Toxicol. 68, 424-429 (1991)), protozoa (BRINKMANN, G.: Z. Wasserund Abwasser-Forsch. 11, 210-215 (1978)), plant protoplasts (OVERMEYER, S. et al.: UWSF-Z. Umweltchem. Ökotox. 6, 5-8 (1994) and green algaes (BRINKMANN, G. u. R.. Kühn: Z. Wasser-und Abwasser-Forsch. 10, 87-98 (1977) were used. Furthermore, it was suggested, to determine mutagenic effects by enterobacteria (ODA, Y. et al.: Mutation Res. 147, 219-229 (1985)) and luminescent bacteria (ULITZUR, S. et al.: Mutation Res. 74, 113-124 (1980)). The methods used before have the disadvantages not to be only time-consuming but also need laboratories with special equipment. Therefore, field investigations were not possible and only single determination of samples were allowed.

### DETAILED DESCRIPTION OF THE PREFERRED EMBODIMENTS

The intention of the present paper was to develop a procedure allowing the determination of noxious substances. For that reason, luminescent bacteria as well as microorganisms were immobilized. The immobilized cells were connected with a light-detecting component to complete a biosensor. The new-developed biosensor is used in a former known flow injection system steering the supply of samples.

The immobilization of luminescent bacteria, natural as well as genetically-engineered, is down in the following procedures, appropriate for microorganisms:

1. the mechanical inclusion of the microorganisms in a measuring chamber with a porous membrane, permeable for pollutants

2. the immobilization in matrices like agar, collagen or polyacrylamide

3. the enclosure in barium—respectively calciumalginate or cellulosesulfate

4. the cohesion on surfaces of membranes or other carrier material, possibly with the help of an appropriate fixing agent like glutaraldehyde or substances with epoxide groups

The light-detecting component of the biosensor is composed of a measuring system, detecting the rather weak emitted radiation by a photodiode for instance, as well as of the corresponding signal pre-amplifier and the indicating device.

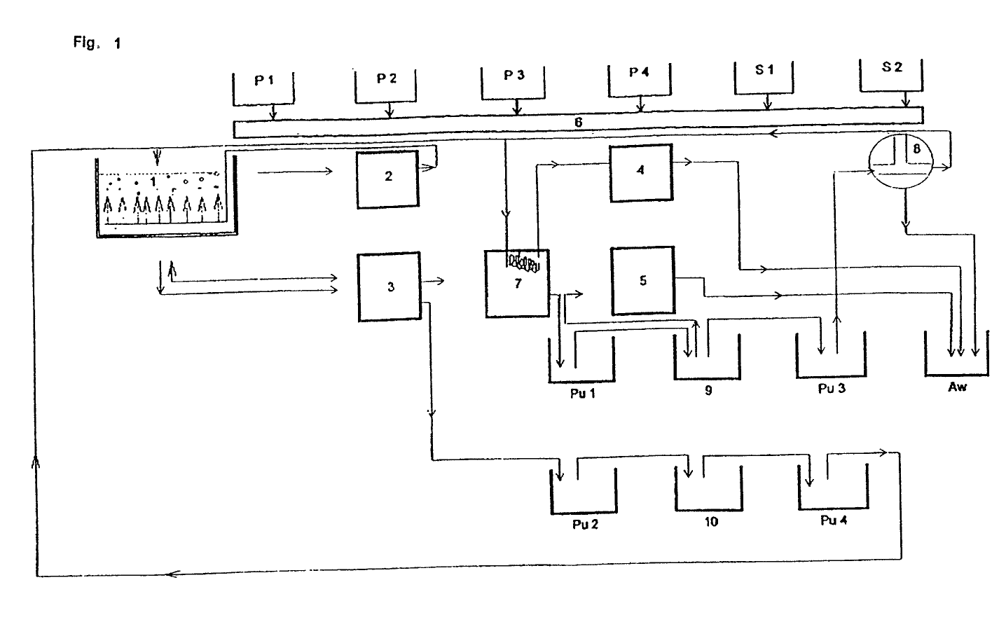

The immobilized cells (the biological component of the biosensor) are incubated in the measuring chamber permeable for light in some parts, on which a light detecting system is located, thus measuring the bioluminescence of the microorganisms. It is also possible to use a darkened measuring chamber, coupled to just one fiber optic, conducting the signals to a light measuring instrument. The supply of the samples is done by the enclosure of the presented biosensor in a former known flow injection system, composed of several pumps and magnetic vents which can be steered by a microprocessor or an external computer (FIG. 1).

The invention presented is a combination of well-known and new-developed features, influencing themselves reciprocal, and, brought together, giving the intended success of the determination of the biological effects of compounds and mixtures of substances over a long time interval for monitoring environmental parameters and controlling processes in agriculture and industry.

The presented use of immobilized luminescent bacteria or bacteria, genetically-engineered to phosphoresce by transfer of the appropriate plasmid-vectors, offers surprisingly new applications facing the common solutions. There are for instance monitoring tasks of environmental parameters, like the control of the air, the surface waters, waste waters or leachate and the control of processes in agriculture and industry. Furthermore, the use of immobilized bacteria allows repeated single measurements. Thereby, the immobilized bacteria experience a manifold use during the investigations of different samples.

Besides the new application in investigating the environmental parameters, a screening of toxicity and mutagenicity for compounds, cosmetics, pharmaceutics, pesticides, partial objects or food is offered.

FIG. 1:

1 chamber with air-saturated carrier-liquid

2 pneumatic-pump

3-5 liquid-pumps

6 6 way magnetic vent

7 injection vent

8 3 way magnetic vent

9 measuring chamber with immobilized cells

10 blank chamber with immobilized cells

P1-P4 sample 1-4

S1-S2 standard 1 and 2

Pu1-Pu4 buffer 1-4

Aw waste water

The invention is supposed to be explained by the following examples:

### MODEL EXAMPLES

### Example 1

The dark mutants of V. fischeri, spontaneously generated, are incubated in a solution of (gl⁻¹) 5 g peptone, 3 g glycerol, 15,5 g NaCl, 0,75 g KCl, 12,3 g MgSO₄×7H₂O, 1,45 g CaCl₂×2H₂O, 0,075 g K₂HPO₄×2H₂O and 1,0 g NH₄ Cl. The needed density of bacteria (around 10⁵ cells ml⁻¹) is determined by extinction and the pH-value is adjusted to 7,0. With the help of a physiological NaCl solution, a natriumalginate solution of 1,2% is made and sterile filtered. Suspension of the bacteria is added, until the end concentration of the natriumalginate solution is 1%. By a syringe fitted to a fine hypodermic needle, and under constant pressure, the described solution is passed drop-wise into a solution of 0,2 M CaCl₂. After solidification of the alginate, the immobilized cells are washed several times in a physiological salt solution. By contact with mutagenic substances, the bioluminescence of the bacteria is enhanced. Thereby, the doubling of the emitted light, opposite to the negative control measurement, is valued as an evidence for the mutagenic effect of a substance. Furthermore, a previous treatment of the sample with a liver enzyme-fraction (S 9-mix), allows the detection of comutagenes.

### Example 2

1,5 g agar is dissolved under warming and stirring in 100 ml distilled water. After cooling down to temperatures of 40-45° C., the suspension of the bacteria V. fischeri (in 30 g NaCl, 6,10 g NaH₂PO₄×3H₂O, 0,204 g MgSO₄×7H₂O, 0,50 g (NH₄)2HPO₄, 3 ml glycerol, 5 g peptone, 0,50 g yeast extract is filled up with H₂O to 1 l⁻¹, adjusted with NaOH or HCl to a pH-value of 7,2±0,2) is mixed to the heated solution. Afterwards, the mixture is solidificated as a sloped agar in the measuring chamber of the flow injection system. By contact with the sample containing toxic substances, the metabolism, and consequently, the bioluminescence, is inhibited. Thereby, a 20% inhibition of the bioluminescence of the luminescent bacteria is evidence for a toxic effect.

### Example 3

5 ml of suspension of V. fischeri (see also example 2) is cooled on ice. In 5 ml of the ice-cooled 0,2 M kaliumphosphate-buffer solution (pH 7,0), 1,63 g acrylamide, 0,08 g N,N-methylene-bisacrylamide and 5 mg ammonia-peroxidsulfate are mixed and stirred until the dissolution is complete. Under consequent stirring, the suspension of bacteria and 0,08 ml N,N,N′,N′-tetramethylethylenediamine are passed drop-wise in the above descripted solution. Polymerization is over after 60 to 90 minutes. The gel is washed with buffer solution and incubated in the measuring chamber of the flow injection system. Like in example 2, the toxic effect of the samples containing compounds is detectable.

### Example 4

E. coli bacteria, genetically-engineered by the transfer of the LUX-gencomplex, as described in example 3, are immobilized and used for the investigations of toxic effects.

### Example 5

4% Na-cellulosesulfate in a solution like in example 1, is passed drop-wise in a 2% solution of polydiallyldimethylammoniumchloride in 0,9% NaCl solution and solidificated for 8 minutes. By transfer of the gained spheres in a hypotonic solution, stabile, light- and pollutantspermeable capsules are build. With the help of the fine hypodermic needle of a syringe , suspensions of the luminescent bacteria like the species V. fischeri, V. harvei, Ph. phosphoreum or Ph. leiognathi or other microorganisms with the ability of bioluminescence, can be insert in the hollow space of the capsules. These capsules are brought into the measuring chamber of the flow injection system to determine the toxic effects of mixtures of substances or single substances.

### Example 6

In suspensions of luminescent bacteria or other microorganisms (example 1 and 5) 2% Na-alginate is dissolved. By dipping sterile glass fiber filters of defined size completely in this solution and by the following incubation of the filter in 0,2 M CaCl₂ solution and several washing steps in 0,9% NaCl solution, stabile glass fiber alginate immobilisates are obtained. The gained immobilisates are inserted into the measuring chamber of the biosensor system.

### Example 7

In a cell suspension, as described in example 1, 4% Na-cellulosesulfate is dissolved completely. By a syringe and through variation of the hypodermic needle diameter, after precipitation in polydiallyldimethylammoniumchloride solution and the washing in 0,9% NaCl solution, stabile cellulosesulfate capsules of different diameters are obtained. This immobilization technique is suitable for different wild species as well as for dark variants of luminescent bacteria and other bacteria with the ability of luminescent.

We claim:

1. The procedure to determine the toxic and mutagenic effects of compounds and mixtures of substances by a biosensor, composed of immobilized cells with the ability of bioluminescence, connected to a light-detecting component.

2. The procedure, following claim 1, characterized by the fact, that luminescent bacteria or genetically-engineered organisms enabled for bioluminescence by the transfer of appropriate plasmid vectors are used.

3. The procedure, following claim 1 and 2, thereby characterized, that the biosensor is a flow system according to FIG. 1.

4. The procedure, following claim 1 to 3, thereby characterized, that it is used for multiple single measurements.

5. The device of a biosensor for the determination of toxic and mutagenic effects of chemicals and mixtures of substances, composed of immobilized, bioluminescent organisms and a light-detecting component.

6. The device, following claim 5, thereby characterized, that the organisms are luminescent bacteria or genetically-engineered organisms enabled for bioluminescence by the transfer of appropriate plasmid vectors.

7. The procedures and the device, following claim 1 to 6, thereby characterized, that the procedures and the device are used for surveillance tasks in the environment, like the control of the air, the surface waters, waste waters or leachate and the control of processes in agriculture and industry.

8. The procedures and the device, following claim 1 to 7, thereby characterized, that the procedures and the device are used for measurements of environmental parameters and for the screening of toxicity and mutagenicity for compounds, cosmetics, pharmaceutics, pesticides, partial objects or food.
